(19) United States
(12) Patent Application Publication
(10) Pub. No.: US 2016/0058358 A1
(43) Pub. Date: Mar. 3, 2016

(54) SYSTEMS AND METHODS FOR DIAGNOSING A BRAIN INJURY

(71) Applicants: Shmuel Marcovitch, Kefar Saba (IL); Michal Marcovitch, Kefar Saba (IL); Shlomi Ben-Ari, Benyamina (IL)

(72) Inventors: Shmuel Marcovitch, Kefar Saba (IL); Michal Marcovitch, Kefar Saba (IL); Shlomi Ben-Ari, Benyamina (IL)

(73) Assignee: Orsan Medical Technologies Ltd.

(21) Appl. No.: 14/844,827

(22) Filed: Sep. 3, 2015

Related U.S. Application Data
(60) Provisional application No. 62/045,080, filed on Sep. 3, 2014.

(51) Int. Cl. A61B 5/00; A61B 5/053

(52) U.S. Cl. CPC A61B 5/4064; A61B 5/053; A61B 5/6814; A61B 5/7275

(57) ABSTRACT

A traumatic brain injury diagnostic system may comprise a current transmission unit, a voltage detection unit, a demodulation unit, and at least one processor. The current transmission unit may output an electric current to at least a first pair of electrodes attached to a head of a subject. The voltage detection unit may detect a voltage via at least a second pair of electrodes attached to the head of the subject proximal to the first pair of electrodes. The demodulation unit may extract an electrical impedance signal from an output electric current of the current transmission unit and the detected voltage of the voltage detection unit. The processor may perform operations to diagnose traumatic brain injury including comparing a static component of the electrical impedance signal to a pre-defined threshold and comparing a dynamic component of the electrical impedance signal to a pre-defined dynamic value.

### CROSS-REFERENCE TO RELATED APPLICATION

This application claims the benefit of priority of U.S. Provisional Patent Application No. 62/045,080, filed on Sep. 3, 2014, the contents of which are incorporated herein by reference in their entirety.

### TECHNICAL FIELD

Embodiments of the present disclosure relate generally to systems and methods provided for detecting and diagnosing a traumatic brain injury and concussion.

### BACKGROUND

Traumatic brain injury (TBI) may present in varying levels of severity, sometimes classified as mild. moderate, and severe. Each classification may include different degrees of damage to the brain.

Traditional means for detecting and diagnosing a traumatic brain injury may include MRI and CT systems. These systems are expensive and are not portable. In addition these systems may fail to detect mild TBI. Mild TBI may be associated with a brain concussion, which is frequently not accompanied by easily detectable symptoms. Many concussions, without additional complications, may be difficult or impossible to detect through traditional means, such MRI or CT scans. Because of the difficulties in detection and diagnosis, concussions may be under diagnosed.

### SUMMARY

Disclosed embodiments include a device configured to diagnose traumatic brain injury. The traumatic brain injury diagnostic device may be configured to receive electrical impedance data from a head of a subject through at least one sensor attached to the head of the subject, and to diagnose a traumatic brain injury based on the electrical impedance data. In some embodiments, the traumatic brain injury diagnostic device may detect and/or diagnose varying levels of traumatic brain injury, such as severe, moderate, or mild. In some, embodiments, the traumatic brain injury diagnostic device may detect and/or diagnose brain concussions. In some embodiments, the traumatic brain injury diagnostic device may perform diagnosis or detection based on static and/or dynamic components of the electrical impedance data.

Some embodiments of the present disclosure include a diagnostic system. The diagnostic system may comprise a current transmission unit configured to output an electric current to at least a first pair of electrodes attached to a head of a subject. The diagnostic system may further comprise a voltage detection unit configured to detect a voltage using at least a second pair of electrodes attached to the head of the subject proximal to the first pair of electrodes. The diagnostic system may also comprise a demodulation unit configured to extract an electrical impedance signal from the output electric current of the current transmission unit and the detected voltage of the voltage detection unit. The diagnostic system may additionally comprise at least one processor. The at least one processor may be configured to diagnose a traumatic brain injury using the electrical impedance signal, and provide an indication of the traumatic brain injury. In some aspects, the traumatic brain injury may comprise a concussion.

In certain aspects, diagnosing the traumatic brain injury using the electrical impedance signal may include comparing a static component of the electrical impedance signal to a pre-defined threshold. In various aspects, diagnosing the traumatic brain injury using, the electrical impedance signal may include comparing a dynamic component of the electrical impedance signal to a pre-defined dynamic value. In certain aspects, the pre-defined dynamic value may be based on at least one of a gender, head circumference, age, weight and height of the subject.

In some aspects, diagnosing the traumatic brain injury using the electrical impedance signal may comprise detecting a trend over a time scale in a range of one minute to twenty-four hours. The trend may be one or more of a trend in a static component of the electrical impedance signal, and a trend in a dynamic component of the electrical impedance signal.

In some aspects, diagnosing the traumatic brain injury using the electrical impedance signal may comprise distinguishing a physiological trend from an artifactual trend by comparing trends in frequency components of the electrical impedance signal.

In some aspects, the output electric current may be a first output electric current, the voltage may be a first voltage, and the electrical impedance signal may be a first electrical impedance signal. The current transmission unit may be further configured to output a second current to at least a third pair of electrodes attached to the head of the subject on a side of the head opposite the first pair of electrodes. The voltage detection unit may be further configured to detect a second voltage using at least a fourth pair of electrodes attached to the head of the subject proximal to the third pair of electrodes. The demodulation unit may be further configured to extract a second electrical impedance signal from the second output electric current of the current transmission unit and the second detected voltage of the voltage detection unit. Diagnosing the traumatic brain injury using the electrical impedance signal may comprise determining a difference. The difference may comprise one or more of a difference between static components of the first electrical impedance signal and static components of the second electrical impedance signal, and a difference between dynamic components of the first electrical impedance signal and dynamic components of the second electrical impedance signal.

In some aspects, the at least one processor may be further configured to receive at least one of an indication, analysis, or numerical value from a blood-test modality for detecting biomarkers of the traumatic brain injury. Diagnosing the traumatic brain injury using the electrical impedance signal may comprise diagnosing the traumatic brain injury using the electrical impedance signal and the at least one of the indication, analysis, or numerical value from the Hood-test modality.

Some embodiments of the present disclosure include a diagnostic method. The diagnostic method may comprise outputting, using a current transmission unit, an electric current to at least a first pair of electrodes attached to a head of a subject. The diagnostic method may also comprise detecting, using a voltage detection unit, a voltage using at least a second pair of electrodes attached. to the head of the subject proximal to the first pair of electrodes. The diagnostic method may further comprise extracting, using a demodulation unit, an electrical impedance signal from the output electric current of the current transmission unit and the detected voltage of the voltage detection unit. The diagnostic method may additionally comprise diagnosing, using at least one processor, a traumatic brain injury using the electrical impedance signal. The diagnostic method may also comprise providing, using the at least one processor, an indication of the traumatic brain injury based on the electrical impedance signal. In some aspects, the traumatic brain injury includes a concussion.

In some aspects, diagnosing the traumatic brain injury may comprise a comparison. The comparison may include one or more of comparing a static component of the electrical impedance signal to a pre-defined threshold, and comparing a dynamic component of the electrical impedance signal to a pre-defined dynamic value. The pre-defined dynamic value may be based on at least one of a gender, head circumference, age, weight and height of the subject.

In some aspects, diagnosing the traumatic brain injury using the electrical impedance signal may comprise detecting a trend over a time scale in a range of one minute to twenty-four hours. The trend may comprise one or more of a trend in a static component of the electrical impedance signal and a trend in a dynamic component of the electrical impedance signal.

In some aspects, diagnosing the traumatic brain injury using the electrical impedance signal may comprise distinguishing a physiological trend from an artifactual trend by comparing trends in frequency components of the electrical impedance signal. In some aspects, the diagnostic method may further comprise outputting, using the current transmission unit, a second current to at least a third pair of electrodes attached to the head of the subject on a side of the head opposite the first pair of electrodes. The diagnostic method may also comprise detecting, using the voltage detection unit, a second voltage using at least a fourth pair of electrodes attached to the head of the subject proximal to the third pair of electrodes. The diagnostic method may further comprise extracting, using the demodulation unit, a second electrical impedance signal from the second output electric current of the current transmission unit and the second detected voltage of the voltage detection unit. The output electric current may be a first output electric current; the voltage may be a first voltage; the electrical impedance signal may be a first electrical impedance signal; and diagnosing the traumatic brain injury using the electrical impedance signal further comprises determining a difference. The difference may comprise one or more of a difference between static components of the first electrical impedance signal and static components of the second electrical impedance signal, and a difference between dynamic components of the first electrical impedance signal and dynamic components of the second electrical impedance signal.

In some aspects, the diagnostic method may further comprise receiving, using the at least one processor, at least one of an indication, analysis or numerical value from a blood-test modality for detecting biornarkers of the traumatic brain injury. Diagnosing the traumatic brain injury using the electrical impedance signal may comprise diagnosing the traumatic brain injury using the electrical impedance signal and the at least one of the indication, analysis or numerical value from the blood-test modality.

Some embodiments of the present disclosure may include a non-transitory computer-readable medium. The non-transitory computer-readable medium may store instructions that, when executed by at least one processor of a diagnostic system, cause the diagnostic system to perform operations. The operations may include outputting an electric current to at least a first pair of electrodes attached to a head of a subject. The operations may also include detecting a voltage using at least a second pair of electrodes attached to the head of the subject proximal to the first pair of electrodes. The operations may further include extracting an electrical impedance signal from the output electric current and the detected voltage. The operations may additionally, include diagnosing a traumatic brain injury using the electrical impedance signal. The traumatic brain injury may include one or more of a traumatic brain injury or a concussion. Diagnosing may comprise a comparison. The comparison may comprise one or more of comparing a static component of the electrical impedance signal to a pre-defined threshold, comparing a dynamic component of the electrical impedance signal to a pre-defined dynamic value. The dynamic value may be based on at least one of a gender, head circumference, age, weight and height of the subject. Diagnosing may comprise detecting a trend over a time scale in a range of one minute to twenty-four hours. The trend may comprise one or more of a trend in the static component of the electrical impedance signal, and a trend in the dynamic component of the electrical impedance signal.

In some aspects, diagnosing the traumatic brain injury using the electrical impedance signal may comprise distinguishing a physiological trend from an artifactual trend by comparing trends in frequency components of the electrical impedance signal.

In some aspects, the operations may further comprise outputting a second current to at least a third pair of electrodes attached to the head of the subject on a side of the head opposite the first pair of electrodes. The operations may additionally comprise detecting a second voltage using at least a fourth pair of electrodes attached to the head of the subject proximal to the third pair of electrodes. The operations may also comprise extracting a second electrical impedance signal from the second output electric current and the second detected voltage. The output electric current may be a first output electric current. The voltage may be a first voltage. The electrical impedance signal may be a first electrical impedance signal. Diagnosing the traumatic brain injury using the electrical impedance signal may further comprise determining a difference. The difference may comprise one or more of a difference between static components of the first electrical impedance signal and static components of the second electrical impedance signal. and a difference between dynamic components of the first electrical impedance signal and dynamic components of the second electrical impedance signal.

In some aspects, the operations may further comprise receiving at least one of an indication, analysis or numerical value from a blood-test modality for detecting biomarkers of the traumatic brain injury. Diagnosing the traumatic brain injury using the electrical impedance signal may comprise diagnosing the traumatic brain injury using the electrical impedance signal and the at least one of the indication, analysis, or numerical value from the blood-test modality.

It is to be understood that both the foregoing general description and the following detailed description are exemplary and explanatory only and are not restrictive of the disclosed embodiments, as claimed.

### BRIEF DESCRIPTION OF THE DRAWINGS

The drawings are not necessarily to scale or exhaustive. Instead, emphasis is generally placed upon illustrating the principles of the subject matter described herein. The accompanying drawings, which are incorporated in and constitute a part of this specification, illustrate several embodiments consistent with the disclosure and together with the description, serve to explain the principles of the disclosure, to the drawings:

FIG. 1 depicts an exemplary embodiment of a device for diagnosing traumatic brain injury.

FIG. 2 depicts a table illustrating impedance recordings of healthy subjects and patients with traumatic brain injury.

FIG. 3 depicts changes in impedance over time in a patient with a traumatic brain injury.

### DETAILED DESCRIPTION

Reference will now be made in detail to exemplary embodiments as with reference to the accompanying drawings, in some instances, the same reference numbers will be used throughout the drawings and the following description to refer to the same or like parts. These embodiments are described in sufficient detail to enable those skilled in the art to practice the disclosed embodiments and it is to be understood that other embodiments may be utilized and that changes may be made without departing from the scope of the disclosed embodiments. The following detailed description, therefore, is not to be interpreted in a limiting sense.

Unless otherwise defined, all technical and/or scientific terms used herein have the same meaning as commonly understood by one of ordinary skill in the art to which the embodiments pertains. Although methods and materials similar or equivalent to those described herein can be used in the practice or testing of the embodiments, exemplary methods and/or materials are described below. In case of conflict, the patent specification, including definitions, will control. In addition, the materials, methods, and examples are illustrative only and are not intended to be necessarily limiting.

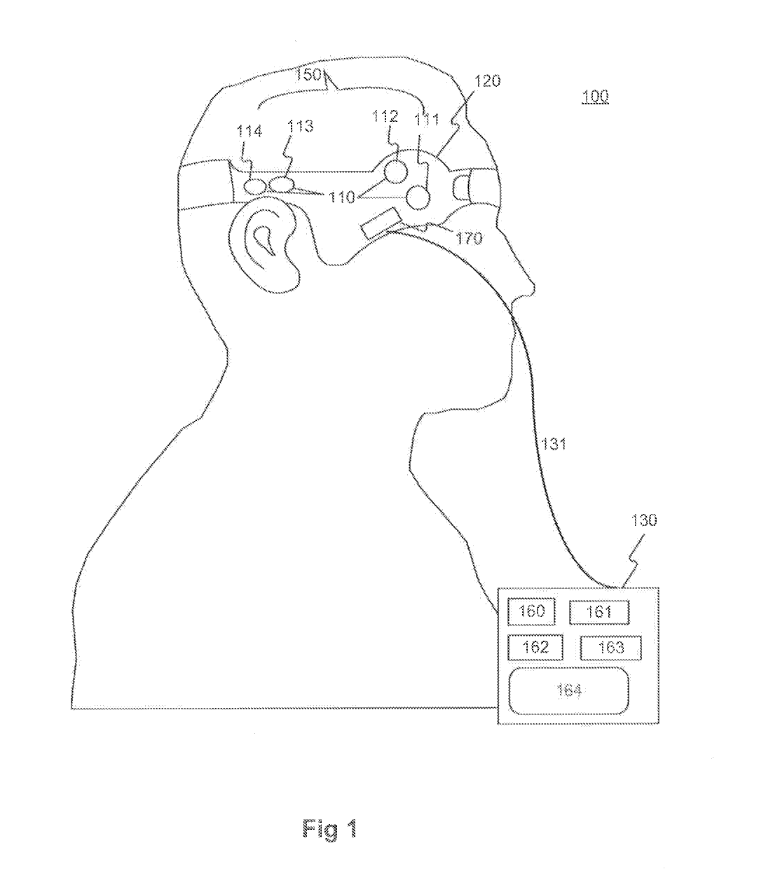

FIG. 1 depicts an exemplary embodiment of a device 100 for diagnosing traumatic brain injury, consistent with disclosed embodiments. The device 100 may include sensors 110 affixed to a subject's head via a headset 120. Sensors 110 may be connected to a diagnostic monitor 130 via wires 131 (or may alternatively include a wireless connection). In some embodiments, sensors 110 may include two electrodes for current delivery and two electrodes for voltage measurement, described in greater detail below.

In some embodiments, diagnostic monitor 130 may include a current transmission unit 161, a voltage detection unit 162, a demodulation unit 163, at least one processor 160, and a display unit 164.

Current transmission unit 161 may be configured to deliver alternating current to the current delivering electrodes. The current may be delivered in a differential (plus-minus) form between the at least two current delivering electrodes and in the frequency range of between a few kHz to hundredths of kHz. An alternating current may have sinusoidal, square-wave or any other appropriate current waveform. Current generation may be implemented by current transmission unit 160 by either a current source designed to produce a stable current with constant amplitude, or by a voltage source. If implemented via a voltage source, current amplitude may not be constant in time as it would depend on the total electrical impedance which may change during the time course the sensors are attached to the subject.

Voltage detection unit 162 may be configured to receive the current received from the voltage receiving electrodes, either through an analog-to-digital component or in an analog fashion.

Demodulation unit 163 may be configured to extract electrical impedance data from the signal obtained by voltage receiving sensors. Demodulation unit 163 may be implemented in an analog or digital form by fast processing hardware such as a field programmable gate array (FPGA) or digital signal processor (DSP). In some embodiments, demodulation unit 163 is configured to receive an additional signal corresponding to the current source, such that the obtained electrical impedance signal will include both amplitude and phase components or real and imaginary components corresponding to the resistive and reactance components of the electrical impedance. If demodulation unit 163 is implemented in an analog fashion, the extracted electrical impedance signal may be sampled by an analog-to-digital component, which may then be transferred to processor 160. If the demodulation is performed digitally, the fast processing hardware may also decimate the extracted signal to provide an electrical impedance signal in a workable sampling rate for processor 160 such as a sampling rate in the range of 100S/sec to a few KS/sec.

The at least one processor 160 may be configured to perform the tasks described above with respect to current transmission unit 161, voltage detection unit 162, and demodulation unit 163. Processor 160 may include several processors, each configured to perform one or more tasks. As used herein, the term “processor” may include an electric circuit that performs a logic operation On an input or inputs. For example, such a processor may include one or more integrated circuits, microchips, microcontrollers, microprocessors, all or part of a central processing unit (CPU), graphics processing unit (CPU), digital signal processors (DSP), field-programmable gate array (FPGA) or other circuit suitable for executing instructions or performing logic operations.

The at least one processor may be configured to perform an action if it is provided with access to, is programmed with, includes, or is otherwise made capable carrying out instructions for performing the action. The at least one processor may be provided with such instructions either directly through information permanently or temporarily maintained in the processor, or through instructions accessed by or provided to the processor. Instructions provided to the processor may be provided in the form of a computer program comprising instructions tangibly embodied on an information carrier, e.g., in a machine-readable storage device, or any tangible computer-readable medium. A computer program may be written in any form of programming language, including compiled or interpreted languages, and it can be deployed in any form, including as a standalone program or as one or more modules, components, subroutines, or other unit suitable for use in a computing environment. The at least one processor may include specialized hardware, general hardware, or a combination of both to execute related instructions. In some embodiments, the at least one processor may include hardware specialized for the task of receiving and interpreting impedance signals; these embodiments are described in more detail below. The at least one processor may also include an integrated communications interface, or a communications interface may be included separate and apart from the at least one processor. The at least one processor may be configured to perform a specified function through a connection to a memory location or storage device in which instructions to perform that function are stored.

Display unit 164 may be employed to output various data to a user of TBI diagnostic device 100. For example, display unit 164 may be configured to display a TBI diagnosis, such as whether TBI is present in a patient, and what the severity (mild, moderate, severe) of the detected TBI is. Display unit 164 may also output any other data collected by TBI diagnostic device 100 during use.

Sensors 110 may be implemented in various configurations. For example, sensor 110 may include at least one electrode configured to deliver alternating current and at least one electrode configured to measure a resulting voltage. In some embodiments, sensors 110 may include two electrodes for current delivery and two electrodes for voltage measurement. In some embodiments, part or all of the at least one voltage receiving electrode and the at least one current delivery electrode may be included in the same physical structure. That is, a single physical electrode may function as both a voltage receiving electrode and as a current delivery electrode. A voltage measurement electrode may be associated with a particular current delivery electrode. A voltage measurement electrode associated with a current delivery electrode may be configured to measure the voltages associated with the current delivered by that particular current delivery electrode. In some embodiments, associated electrodes may be located close or in substantially the same place as one another on a patient. In other embodiments, associated electrodes may be located remotely from each other on a patient.

Consistent with some disclosed embodiments, the at least one processor may be configured to receive a signal from sensors 110. As used herein, a signal may include any time-varying or spatially-varying quantity. Receiving a signal may include obtaining a signal through conductive means, such as wires or circuitry; reception of a wirelessly transmitted signal; and/or reception of a signal previously recorded, such as a signal stored in memory. Receiving a signal may further encompass other methods known in the art for signal reception. In some embodiments, a received signal or signals may include impedance data.

Processor 160 may be configured to receive and analyze one or more signals associated with a brain of a subject and may be included in diagnostic monitor 130, as part of exemplary TBI diagnostic device 100. Processor 160 may be configured to perform all or some of the signal analysis methods described herein, or some of those functions may be performed by a separate processor. Processor 160 may also be configured to perform any common signal processing task known to those of skill in the art, such as filtering, noise-removal, etc. Processor 160 may further be configured to perform pre-processing tasks specific to the signal analysis techniques described herein. Such pre-processing tasks may include, but are not limited to, removal of signal artifacts, such as motion artifacts.

Processor 160 may be configured to receive a signal from one or more sensors 110, included in exemplary headset 120 of FIG. 1. Sensors 110 may be arranged singly, in pairs, or in other appropriate groupings, depending on implementation. The sensors on exemplary headset 120 may be arranged so as to obtain signals including impedance data. Impedance data may be measured by one or two sensor sections 150. If two sections 150 are used, they may be disposed on the right and left sides of the head to correspond with the right and left hemispheres of the brain, for example. While only one sensor section 150 is shown in FIG. 1, an opposite side of the subject's head might include a similar electrode arrangement. In addition, each sensor section 150 may include one pair of front electrodes, front current electrode 111 and front voltage electrode 112, and one pair of rear electrodes, rear current electrode 114, and rear voltage electrode 113. The distance between the pairs may be adjusted such that a particular aspect of an intracranial physiological condition is satisfied. In some embodiments, headset 120 may be adapted to maintain a specific distance between any or all of sensors 110. The electrode configuration depicted in FIG. 1 is only one example of a suitable electrode configuration. Additional embodiments may include more or fewer sensors 110, additionally or alternatively arranged in different areas of exemplary headset 120. Other embodiments may include sensors 110 configured on an alternatively shaped headset to reach different areas of the subject's head as compared to the exemplary headset 120. In some embodiments, headset 120 may include multiple unconnected portions.

Pairs of sensors 110 may include a current output electrode and a voltage input electrode. For instance, front current electrode 111 and front voltage electrode 112 may form an electrode pair. In one embodiment, an output current may be generated by diagnostic device monitor 130 and passed between front current electrode 111 and rear current electrode 114, or vice versa. The output current may include an alternating current (AC) signal of constant amplitude and stable frequency in the range of 1 kHz to 1 MHz. In some embodiments, a frequency between 50 kHz and 100 kHz may be used. An input voltage resulting due to the output current may be measured between front voltage electrode 112 and rear voltage electrode 113. An input voltage may be measured at the same frequency as the output current. A comparison between the output current signal, e.g. a measurement signal, and the input voltage signal, e.g. a response signal, may be used to extract impedance data from the subject. More specifically, a magnitude of the bioimpedance may be computed as a ratio of the voltage signal amplitude to the current amplitude signal, and a phase of the bioimpedance may be computed as the phase difference by which the voltage signal leads the current signal. Additional impedance components may be computed from the current signal and the voltage signal, or from the bioimpedance magnitude and phase, as required. In embodiments that use two sensor sections 150, each sensor section 150 may receive an output current signal at different frequencies to help prevent interference. Frequencies may differ by as much as 5-10 kHz or as little as 50-100 Hz.

An impedance signal may also include output current at more than a single AC frequency. The output current may include a set of predefined frequencies and amplitudes, for example in the range of 1 kHz to 1 MHz, with detection of the measured voltage at all of the frequencies or a part of the frequency range.

Blood and fluid flow into and out of the head, and more specifically, the brain, may result in changes in the cranial bioimpedance characterized by impedance data extracted from the signal received by sensors 110. Bioimpedance changes may correlate with blood volume and blood pressure in the head and brain, as well as the volumes and pressure of other fluids within the brain. The cardiac cycle, respiration cycle, and slow-wave autoregulation cycles may affect the volume and pressure of both blood and other fluids in the brain. Injury to the head, which may result in the pooling of blood and/or fluid in the head, may also affect impedance measurements. In general, because blood and other fluids have relatively low impedance when compared with tissue found in the head, higher blood or fluid volume results in a lower impedance magnitude. Impedance changes associated with differing blood and fluid volume and pressure within the brain may also cause variations in the frequency response of the brain impedance. Analysis of impedance measurements at different frequencies and on differing timescales may provide information useful for diagnosis of traumatic brain injury.

Processor 160 may receive electrical impedance data extracted from signals received by TBI diagnostic device 100. The at least one processor 160 may further receive additional and/or ancillary inputs, as explained in greater detail below. Processor 160 may analyze the electrical impedance data to detect the presence of TBI and to diagnose the severity of TBI. In some embodiments, processor 160 may detect and diagnose brain concussions.

The exemplary headset 120 may further include various circuitry 170 for signal processing or other applications and may include the capability to transmit data wirelessly to diagnostic monitor 130 or to other locations. In an additional embodiment, diagnostic monitor 130 may be integrated with headset 120.

Exemplary headset 120 may include various means for connecting, encompassing, and affixing sensors 110 to a patient's head. For example, headset 120 may include two or more separate sections that are connected to form a loop or a band that circumscribes the patient's head. Bands, fasteners, electrode holders, wiring, hook-and-loop connector strips, buckles, buttons, clasps, etc. may be used and adjustable in order to fit headset 120 to a patient's head. Portions of exemplary headset 120 may be substantially flexible and portions of the exemplary headset 120 may be substantially inflexible. For example, electrode-including portions of exemplary apparatus 120 may be substantially inflexible in order to, among other things, substantially fix sensors 110 in specific anatomical positions on the patient's head. In addition to or in the alternative, other portions, such as bands or connectors holding the exemplary headset 120 to a patient's head, may be substantially flexible, elastic and/or form fitting.

Any portion of exemplary headset 120 may be specifically designed, shaped or crafted to fit a specific or particular portion of the patient's anatomy. For example, portions of exemplary headset 120 may be crafted to fit near, around or adjacent to the patient's ear. Portions of exemplary headset 120 may be specifically designed, shaped or crafted to fit the temples, forehead and/or to position sensors 110 in specific anatomical or other positions. Portions of the exemplary headset 120 may be shaped such that sensors 110 for other included measurement devices) occur in specific positions for detecting characteristics of blood and fluid flow in the head or brain of the patient. Examples of such blood flow may occur in any of the blood vessels discussed herein, such as the arteries and vasculature providing blood to the head and/or brain, regardless of whether the vessels are in the brain or feed the brain.

Exemplary headset 120 may include features suitable for improving comfort of the patient and/or adherence to the patient. For example exemplary headset 120 may include holes in the device that allow ventilation for the patient's skin. Exemplary headset 120 may further include padding, cushions, stabilizers, fur, foam felt, or any other material for increasing patient comfort.

As mentioned previously, exemplary headset 120 may include one or more additional sensors. In addition to or as an alternative to electrical or electrode including devices for measuring bioimpedance. Additional sensors 140 may comprise any other suitable devices, and are not limited to the single sensor illustrated in FIG. 1. Other examples of additional sensor 140 include devices for measuring local temperature (e.g., thermocouples, thermometers, etc.) and/or devices for performing other bio measurements and for devices for measuring movement and positioning of the patient (e.g., accelerometers and/or inclinometers).

Exemplary headset 120 may include any suitable form of communicative mechanism or apparatus. For example, headset 120 may be configured to communicate or receive data, instructions, signals or other information wirelessly to another device, analytical apparatus and/or computer. Suitable wireless communication methods may include radiofrequency, microwave, and optical communication, and may include standard protocols such as Bluetooth, WiFi, etc. In addition to, or as an alternative to these configurations, exemplary headset 120 may further include wires, connectors or other conduits configured to communicate or receive data, instructions, signals or other information to another device, analytical apparatus and/or computer. Exemplary headset 120 may further include any suitable type of connector or connective capability. Such suitable types of connectors or connective capabilities may include any standard computer connection (e.g., universal serial bus connection, firewire connection, Ethernet or any other connection that permits data transmission). Such suitable types of connectors or connective capabilities may further or alternatively include specialized ports or connectors configured for the exemplary apparatus 100 or configured for other devices and applications.

Electrical impedance data obtained by TBI diagnostic device 100 may include both static and pulsating components. The amplitude of the pulsating components may be three orders of magnitude smaller than the static components. For example, static electrical impedance components may be measured between 50 and 200 ohms, depending on the health of the patient. Pulsating components, however, may be measured as a milliohm level signal overlaid on the static signal. As used herein. “pulsating components” are those components of the electrical impedance that experience fluctuation with consistently occurring physiological cycles, such as cardiac cycles, respiratory cycles, and slow-wave autoregulation cycles. As used herein, “static components” are those components that are not associated with a consistently recurring physiological phenomenon, such as a cardiac cycle or respiratory cycle. The static component of the electrical impedance may represent a baseline impedance, with which the pulsatile components are overlaid. Static components, as described herein, are not fixed, and may change over time. Static components of electrical impedance data may change quickly and may Change slowly. For example, the occurrence of a stroke or a traumatic brain injury may cause static electrical impedance components to express a large change in a short amount of time. The aftermath of a stroke or a traumatic brain injury may cause slower changes in the static electrical impedance, as the physiological state of the brain slowly adjusts and reacts to the adverse event.

Both the static and the pulsating components of the cerebral electrical impedance may provide physiological informative data. The pulsating signal may include information on cerebral hemodynamics including cardiac pulse, respiration and slower-wave physiological attenuations. The static component may contain information on the tissues through which electric current flows. When obtained through a tetra-polar electrode configuration (wherein there are 4 electrodes, and the current and voltage electrodes are separate), the static component of an electrical impedance signal may be relatively free of effects produced by the interface between the electrodes and the body (e.g., effects produced by the skin and/or by the electrode/skin interface). In contrast, a bipolar electrode configuration (in which the current and voltage electrodes are identical), may also be influenced by tissue through which the electric current penetrates in the body (e.g. skin) as well as the electrode-surface electrical impedance.

The static component of the electrical impedance in the tetra-polar configuration may be influenced from the distance between the electrodes and their spatial outline, the gender of the subject, and the amount of fluid, both intracellular and extracellular in the medium through which the current flows. In the case of impedance measurements done on the scalp, current flows through the brain, and the static component of the electrical impedance may be heavily influenced by the amount of extra-cellular fluid in the brain.

A traumatic brain injury may decrease dramatically the static component of the cerebral electrical impedance due to at least two factors. First, the rapid accumulation of extracellular fluid immediately after TBI onset may cause a decrease in the static component of the electrical impedance. Second, the disruption of the blood-brain-barrier may also cause a decrease in the static component of the electrical impedance. In a non-pathological scenario, the blood-brain-barrier obstructs electric current and serves both as a biochemical blockage, and as a blockage to electric current due its dense endothelium tissue structure. In the event of TBI, even mild, a blood-brain-barrier disruption occurs and immediately decreases the static component of the cerebral electrical impedance.

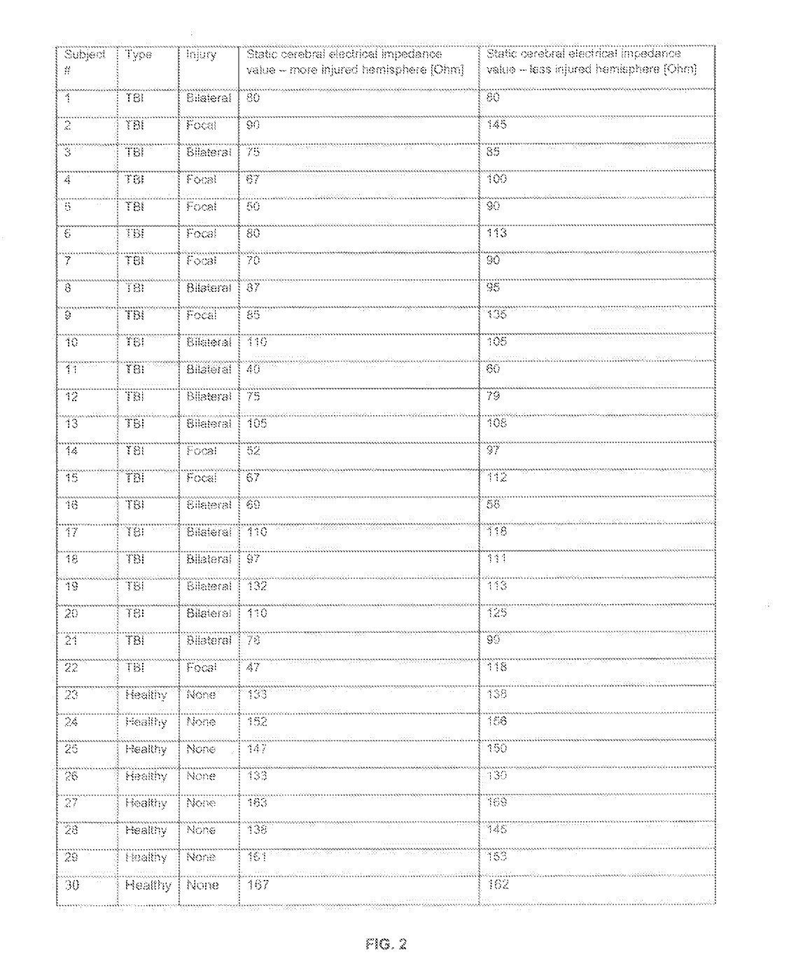

As an exemplary illustration, FIG. 2 is a table showing the mean static cerebral electrical impedance measures of both injured and less injured sides in 22 TBI patients as well as 8 healthy volunteers presented. As can readily be seen from the table, there is a marked difference between the static electrical impedance measures taken from healthy and pathological brain hemispheres. Thus, by comparing a static electrical impedance measurement to a predefined threshold value, TBI may be detected and a level of TBI diagnosed.

A predefined threshold value may be determined in several ways. In some embodiments, a patient may undergo a pre-screening test when healthy that sets a personal threshold value of static electrical impedance. Such a pre-screen may be valuable for patient's that expect to encounter the possibility of TBI, such as soldiers, athletes, construction workers, elderly patients, etc. in alternative embodiments, a threshold may be determined for a particular patient that has not been pre-screened based on physiological factors such as age, gender, height, head circumference, and weight. In further embodiments, a standard threshold may be applied to all patients, regardless of physiology or history. In some embodiments, a standard threshold may be between 120 and 130 ohms, and may include a threshold at 125, 126, and/or 127 ohms. In some embodiments, multiple predetermined thresholds may be used to determine the degree of TBI that has occurred. Because a natural value of static electrical impedance may differ between healthy individuals, in some embodiments, predetermined thresholds may include ranges for which a probability of TBI is determined. For example, one impedance reading may indicate a 25% chance of TBI occurrence, and a slightly higher reading may indicate a 50% chance, and so on. In such embodiments, additional information or further observation may be used to finalize a TBI detection or diagnosis.

In some embodiments, additional information may be used to detect and diagnose TBI. Additional information helpful for the detection and diagnosis may further be obtained by comparing the static electrical impedance measurements between a healthy and a non-healthy side of a patient's head. If the difference between the two measurements, when one hemisphere is known to be healthy, exceeds a predefined symmetry threshold value, it may be determined that TBI has occurred and to what degree. In some embodiments, a predefined symmetry threshold may be between 15 and 20 ohms, and may include a threshold of 17, 18, and/or 19 ohms.

An increasing trend of the static electrical impedance value may follow the sudden decrease in the static electrical impedance value immediately after TBI onset. The increasing trend of the static electrical impedance value may occur over a period of hours to days after TBI onset. The increasing trend of the static electrical impedance value may occur until the static electrical impedance value is restored to the original non-pathological value. This trend may be the result of several physiological occurrences. First, the evacuation of extra-cellular fluids to the vascular system or during the recovery process may serve to increase the static cerebral electrical impedance. Second, the evacuation of extra-cellular fluids into intra-cellular space during an edema formation process may serve to increase the static cerebral electrical impedance. Finally, the repair of the blood brain barrier may also serve to increase the static cerebral electrical impedance. Each of these factors may contribute to an increase in the static value of the cerebral electrical impedance. However, a restoration of the static value of the cerebral electrical impedance to pre-trauma levels may not be indicative of a fully healed injury.

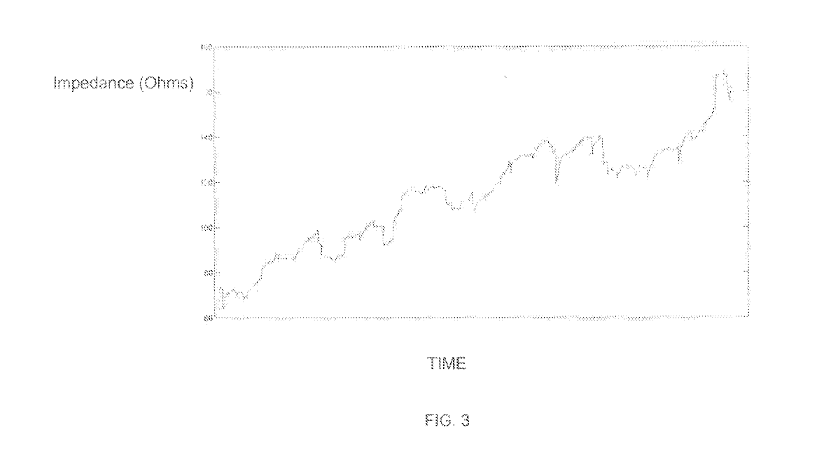

FIG. 3 is a graph illustrating the change in the static impedance over time in a patient that has suffered from a TEL The time axis in FIG. 3 corresponds to a 7 day post trauma period. As illustrated, there is a significant increase in the static component of the cerebral electrical impedance by a factor of more than two during the time period. The trending characteristic of the static component of the cerebral electrical impedance signal in patients who suffered from TBI after TBI onset may provide an additional marker to distinguish these patients from subjects with no brain injury. For example, in patients that display a static impedance at or near the predetermined threshold, or within a threshold range that does not indicate a certainty of TBI, further monitoring of the impedance trend may provide the additional information required by a physician or by TBI diagnostic device 100 to make an accurate detection and or diagnosis of TBI. Thus, the post-trauma trend characteristic may be a source of additional information to detect and diagnose TBI.

The trending characteristic may be especially important in the case of mild TBI injuries, such as concussions, in which the decrease in the static value of the cerebral electrical impedance may not be sufficiently strong to produce a certainty of TBI. By monitoring the post-trauma trend, for example by slope detection performed over a time course of a few minutes to several hours, a more certain diagnosis of TBI may be provided.

In some embodiments, processor 160 may be configured to analyze a trending characteristic to determine whether it represents a physiological trend or whether it is merely an artifact. In some embodiments, the processor may be configured to analyze a trending characteristic over a period ranging from one minute to twenty four hours. Sensor artifacts, such as those due to sweating or fever, and system artifacts, such as those due to warming, may introduce a trend in the static electrical impedance component with apparent similarity to that appearing due to TBI pathology. Processor 160 may be configured to distinguish between real electrical impedance trending and artifact drifting based on various criteria. For example, the criteria may be based on an expected behavior of the real and imaginary components (or amplitude and phase components) of the electrical impedance in case of physiological trending which may be different or absent in case of sensor artifacts. In addition, a comparison of the trending behavior determined at different current output frequencies may provide an additional distinction between physiological trending and artifact drifting.

In some embodiments, processor 160 may further be configured to analyze and compare impedance signals obtained via different sensor configurations to detect and diagnose TBI. By comparing pulsatile and/or static parameters of the cerebral electrical impedance signals between different sensor configurations, or between sensor configurations corresponding to the more injured hemisphere and the less injured hemisphere, or between direct sensor configuration and a crossed sensor configuration (e.g., combining current and voltage electrodes from different sensor sets), a more accurate determination of TBI may be made.

When analyzing a static component of cerebral electrical impedance, processor 160 may be configured to consider various parameters. For example, processor 160 may be configured to consider any combination of the following factors: the mean of the static component, the slope over time, the standard deviation, the kurtosis, and any other mathematical operations commonly used by the community in the art of signal processing.

Some embodiments consistent with the present disclosure include additional information obtained via analysis, by the at least one processor, of pulsatile components of the electrical impedance. For example, cerebral electrical impedance parameters corresponding to the pulsatile component include any combination of pulse amplitude, area-under-the curve, maximal and minimal derivative values and their timings. Cerebral electrical impedance parameters may further include the P1, P2, and P3 features of the intracranial waveform, which are also shadowed in the electrical impedance waveform and the notches N1, N2, and N3 between them, including their timing, amplitude, first derivative value, second derivative value and curvature.

The cerebral electrical impedance parameters above may be extracted from the amplitude, phase, real value, imaginary value or any other mathematical operation corresponding to a functional of cerebral electrical impedance signals corresponding to a certain sensor configuration, a combination of sensor configurations, a crossed sensor configuration, or any combination of sensor configurations and crossed sensor configurations.

In some embodiments of the present disclosure may, processor 160 may use any combination of the parameters discussed above, Multi-parameters analysis may enable TBI diagnostic device 100 to provide more accurate detection and or diagnosis of TBI, mild, moderate, and severe. In some embodiments, a user of TBI diagnostic device 100 may have access to each of the parameters discussed herein, and may configure TBI diagnostic device 100 to detect and or diagnose TBI based on a user-selected subset of these parameters.

In further embodiments, TBI diagnostic device 100 may be configured to receive data from external sources in order to supplement the analysis of potential TBI. For example, it has been shown that, due to disruption of the blood brain barrier, antigenic protein S100B may leak into the blood serum, triggering an increase in S100B antibodies. Detection of these antibodies may provide valuable additional information in detecting and diagnosing TBI. TBI diagnostic device 100 may be configured to receive data regarding S100B antibody counts in the blood. Other biomarkers appearing in blood tests may also be used. Further additional information may, for example, be provided by EGG and/or arterial blood pressure signals.

The foregoing detailed description of the drawings, and of the associated embodiments, has been presented for purposes of illustration only. This description is not exhaustive and does not limit the claimed subject matter to the precise form disclosed. Those skilled in the art will appreciate from the foregoing description that modifications and variations are possible in light of the above teachings or may be acquired from practicing the disclosed embodiments. For example, the steps described need not be performed in the same sequence discussed or with the same degree of separation. Likewise various steps may be omitted, repeated, or combined, as necessary, to achieve the same or similar objectives. Similarly, the systems described need not necessarily include all parts described in the embodiments, and may also include other parts not describe in the embodiments. Accordingly, the claimed subject matter is not limited to the above-described embodiments, but instead is defined by the appended claims in light of their full scope of equivalents.

What is claimed is:

1. A diagnostic system, comprising:
a current transmission unit configured to output an electric current to at least a first pair of electrodes attached to a head of a subject;
a voltage detection unit configured to detect a voltage using at least a second pair of electrodes attached to the head of the subject proximal to the first pair of electrodes;
a demodulation unit configured to extract an electrical impedance signal from the output electric current of the current transmission unit and the detected voltage of the voltage detection unit; and
at least one processor configured to:
diagnose a traumatic brain injury using the electrical impedance signal, and
provide an indication of the traumatic brain injury.

2. The diagnostic system of claim 1, wherein the traumatic brain injury includes a concussion.

3. The diagnostic system of claim 1, wherein diagnosing the traumatic brain injury using the electrical impedance signal comprises comparing a static component of the electrical impedance signal to a pre-defined threshold.

4. The diagnostic system of claim 1, wherein diagnosing the traumatic brain injury using the electrical impedance signal comprises comparing a dynamic component of the electrical impedance signal to a pre-defined dynamic value.

5. The diagnostic system of claim 4, wherein the pre-defined dynamic value is based on at least one of a gender, head circumference, age, weight, and height of the subject.

6. The diagnostic system of claim 1, wherein diagnosing the traumatic brain injury using the electrical impedance signal comprises detecting, over a time scale in a range of one minute to twenty-four hours, one or more of:
a trend in a static component of the electrical impedance signal, and
a trend in a dynamic component of the electrical impedance signal.

7. The diagnostic system of claim 1, wherein diagnosing the traumatic brain injury using the electrical impedance signal comprises distinguishing a physiological trend from an artifactual trend by comparing trends in frequency components of the electrical impedance signal.

8. The diagnostic system of claim 1, wherein:
the output electric current is a first output electric current;
the voltage is a first voltage;
the electrical impedance signal is a first electrical impedance signal;
the current transmission unit is further configured to output a second current to at least a third pair of electrodes attached to the head of the subject on a side of the head opposite the first pair of electrodes;
the voltage detection unit is further configured to detect a second voltage using at least a fourth pair of electrodes attached to the head of the subject proximal to the third pair of electrodes;
the demodulation unit is further configured to extract a second electrical impedance signal from the second output electric current of the current transmission unit and the second detected voltage of the voltage detection unit; and
wherein diagnosing the traumatic brain injury using the electrical impedance signal comprises determining one or more of:
a difference between static components of the first electrical impedance signal and static components of the second electrical impedance signal, and
a difference between dynamic components of the first electrical impedance signal and dynamic components of the second electrical impedance signal.

9. The diagnostic system of claim 1, wherein the at least one processor is further configured to receive at least one of an indication, analysis, or numerical value from a blood-test modality for detecting biomarkers of the traumatic brain injury, and wherein diagnosing the traumatic brain injury using the electrical impedance signal comprises diagnosing the traumatic brain injury using the electrical impedance signal arid the at least one of the indication, analysis, or numerical value from the blood-test modality.

10. A diagnostic method, comprising:
outputting, using a current transmission unit, an electric current to at least a first pair of electrodes attached to a head of a subject;
detecting, using a voltage detection unit, a voltage using at least a second pair of electrodes attached to the head of the subject proximal to the first pair of electrodes;
extracting, using a demodulation unit, an electrical impedance signal from the output electric current of the current transmission unit and the detected voltage of the voltage detection unit;
diagnosing, using at least one processor, a traumatic brain injury using the electrical impedance signal; and
providing, using the at least one processor, an indication of the traumatic brain injury based on the electrical impedance signal.

11. The method of claim 10, wherein the traumatic brain injury includes a concussion.

12. The method of claim 10, wherein diagnosing the traumatic brain injury comprises one or more of:
comparing a static component of the electrical impedance signal to a pre-defined threshold, and
comparing a dynamic component of the electrical impedance signal to a pre-defined dynamic value based on at least one of a gender, head circumference, age, weight, and height of the subject.

13. The method of claim 10, wherein diagnosing the traumatic brain injury using the electrical impedance signal comprises detecting, over a time scale in a range of one minute to twenty-four hours, one or more of
a trend in a static component of the electrical impedance signal, and
a trend in a dynamic component of the electrical impedance signal.

14. The method of claim 10, wherein diagnosing the traumatic brain injury using the electrical impedance signal comprises distinguishing a physiological trend from an artifactual trend by comparing trends in frequency components of the electrical impedance signal.

15. The method of claim 10, further comprising:
outputting, using the current transmission unit, a second current to at least a third pair of electrodes attached to the head of the subject on a side of the head opposite the first pair of electrodes;
detecting, using the voltage detection unit, a second voltage using at least a fourth pair of electrodes attached to the head of the subject proximal to the third pair of electrodes;
extracting, using the demodulation unit, a second electrical impedance signal from the second output electric current of the current transmission unit and the second detected voltage of the voltage detection unit; and
wherein the output electric current is a first output electric current, the voltage is a first voltage, the electrical impedance signal is a first electrical impedance signal, and diagnosing the traumatic brain injury using the electrical impedance signal further comprises determining one or more of:
a difference between static components of the first electrical impedance signal and static components of the second electrical impedance signal, and
a difference between dynamic components of the first electrical impedance signal and dynamic components of the second electrical impedance signal.

16. The method of claim 10, further comprising receiving, using the at least one processor, at least one of an indication, analysis, or numerical value from a blood-test modality for detecting biomarkers of the traumatic brain injury, and wherein diagnosing the traumatic brain injury using the electrical impedance signal comprises diagnosing the traumatic brain injury using the electrical impedance signal and the at least one of the indication, analysis, or numerical value from the blood-test modality.

17. A non-transitory computer-readable medium storing instructions that, when executed by at least one processor of a diagnostic system, cause the diagnostic system to perform operations of:
outputting an electric current to at least a first pair of electrodes attached to a head of a subject;
detecting a voltage using at least a second pair of electrodes attached to the head of the subject proximal to the first pair of electrodes;
extracting an electrical impedance signal from the output electric current and the detected voltage;
diagnosing a traumatic brain injury using the electrical impedance signal, the traumatic brain injury includes one or more eta traumatic brain injury and a concussion, and the diagnosing comprises one or more of:
comparing a static component of the electrical impedance signal to a pre-defined threshold.
comparing a dynamic component of the electrical impedance signal to a pre-defined dynamic value based on on at least one of a gender, head circumference, age, weight, and height of the subject; and
detecting, over a time scale in a range of one minute to twenty-four hours, one or more of:
a trend in the static component of the electrical impedance signal, and
a trend in the dynamic component of the electrical impedance signal.

18. The computer-readable medium of claim 17, wherein diagnosing the traumatic brain injury using the electrical impedance signal comprises distinguishing a physiological trend from an artifactual trend by comparing trends in frequency components of the electrical impedance signal.

19. The computer-readable medium of claim 17, the operations further comprising:
outputting a second current to at least a third pair of electrodes attached to the head of the subject on a side of the head opposite the first pair of electrodes;
detecting a second voltage using at least a fourth pair of electrodes attached to the head of the subject proximal to the third pair of electrodes;
extracting a second electrical impedance signal from the second output electric current and the second detected voltage; and
wherein the output electric current is a first output electric current, the voltage is a first voltage, the electrical impedance signal is a first electrical impedance signal, and diagnosing the traumatic brain injury using the electrical impedance signal further comprises determining one or more of:
a difference between static components of the first electrical impedance signal and static components of the second electrical impedance signal, and
a difference between dynamic components of the first electrical impedance signal and dynamic components of the second electrical impedance signal.

20. The computer-readable medium of claim 17, the operations further comprising:
receiving at least one of an indication, analysis or numerical value from a blood-test modality for detecting biomarkers of the traumatic brain injury, and
wherein diagnosing the traumatic brain injury using the electrical impedance signal comprises diagnosing the traumatic brain injury using the electrical impedance signal and the at least one of the indication, analysis, or numerical value from the blood-test modality.
